(19) United States
(12) Patent Application Publication
(10) Pub. No.: US 2007/0248621 A1
(43) Pub. Date: Oct. 25, 2007

(54) Food product containing policosanols

(75) Inventors: Stuart Lowther, Ontario (CA); John Sbordone, Stamford, CT (US)

Correspondence Address: OCCHIUTI ROHLICEK & TSAO, LLP, 10 FAWCETT STREET, CAMBRIDGE, MA, 02138, US

(73) Assignee: METABEV LLC

(21) Appl. No.: 11/410,317

(22) Filed: Apr. 24, 2006

(51) Int. Cl. A61K 36/899; A61K 36/8962; A61K 36/06; A61K 31/7012; A61K 31/56; A61K 31/525; A61K 31/015; A61K 36/68

(52) U.S. Cl. 424/195.16; 424/750; 424/754; 424/738; 514/171; 514/251; 514/560; 514/23; 514/53; 514/763

(57) ABSTRACT

The invention provides a novel composition and method for reducing “bad” cholesterol and increasing “good” cholesterol. The composition of the invention comprises a food product having sugar cane juice. The sugar can juice preferably contains policosanols. A method of preventing or treating cardiovascular disease comprising administering a composition comprising sugar cane juice is also provided.

### FIELD OF INVENTION

The present invention relates to food products containing natural ingredients that promote health, particularly cardiovascular health.

### BACKGROUND OF THE INVENTION

Cardiovascular disease is a leading cause of mortality and morbidity in industrialized nations. Elevated low density lipoprotein-cholesterol (LDL) levels is a major risk factor for cardiovascular disease. While various pharmaceutical agents have been developed that lower cholesterol, there are increasing concerns regarding their use. For example, the popular cholesterol lowering drug, Baycol (cerivastatin) was withdrawn from the market after numerous deaths were associated with its use. As a result, there is an increasing demand for alternatives to prescription drugs. Several natural supplements, such as niacin, inositol hexaniacinate, gugulipid and pantethine have been shown to be effective in lowering cholesterol levels. Niacin has been shown to lower total cholesterol and LDL cholesterol while at the same time raising the level of the “good” HDL cholesterol. However, niacin can cause undesirable side effects such as skin flushing, stomach irritation, nausea, glucose intolerance, visual disturbances and liver damage. Gugulipid, also known as gugul is an extract from the myrrh tree. Gugulipid increases the breakdown of LDL cholesterol in the liver thereby lowering LDL levels. It has also been shown to increase HDL cholesterol. A side effect of using gugulipid extract, however, is mild abdominal discomfort. Pantethine is a form of pantothenic acid (vitamin B5) that has also been shown to lower cholesterol. It reduces levels of LDL and total cholesterol, and raises HDL levels. Pantethine appears to work by improving fat metabolism and by slowing down production of cholesterol in the liver.

Another natural product that has been shown to be very effective in reducing LDL and total cholesterol levels is policosanol. Policosanol is a mixture of higher primary aliphatic alcohols. It consists mainly of octacosanal (CH₃—CH_{2 (26)}—CH₂—OH), triacontanol, and hexacosanol. It also includes other alcohols, namely tetracosanol, heptaconsanol, nonacosanol, dotriacontanol, and tetratriacontanol. There are several medical studies showing that policosanol can reduce both total cholesterol and LDL (“bad”) cholesterol by about 10-20%. Some studies also suggest that treatment with policosanol results in an elevation of HDL (“good”) cholesterol. There is some evidence that policosanol may inhibit coenzyme A (HMG-CoA) reductase. In addition, LDL binding, internalization, and degradation are significantly increased after policosanol treatment. Policosanol is typically derived from sugar cane wax, although it may also be derived from beeswax. The constituent alcohols may vary depending on the source of the policosanol.

The beneficial effects of policosanol on cholesterol levels have been recognized for some time. U.S. Pat. No. 5,952,393 is directed to a composition for reducing serum cholesterol in humans and animals. The composition includes phytosterol and policosanol in a tablet or soft gelatin capsule formulation. The policosanol is derived from rice bran wax. U.S. Pat. No. 6,565,896 discloses a cholesterol treatment composition comprising: Green Tea extract, Gamma Oryzanol, Gugulipids, Beta Sitosterol, Artichoke extract, Grape Seed extract, Chromium, Pantethine, and Policosanol. United States Patent Application US 2005/0002992 discloses a food or beverage that contains a human metabolic supplement that will enhance human metabolism. The supplement comprises ingredients selected from the group consisting of policosanol, aspartate, lipoic acid, huperzine, trimethylglycine, and medium chain tryglycerides. The policosanol used in the food or beverage is an extracted, dried supplement derived from sugar cane wax.

In addition to improving serum lipid profiles, policosanol also reduces several other cardiovascular disease risk factors. It has been shown to reduce LDL oxidation, platelet aggregation, endothelial cell damage, and smooth muscle proliferation. It is also effective in patients with Intermittent claudication. Policosanol has also been shown to have beneficial effects on other diseased states such as type 2 diabetes, prostate cancer, and inflammation. It has also been shown to promote vascular wall function and structure as well as the function of nerve growth factors and neurotrophic factors. Policosanol can also be used as an agent to suppress toxic metal ionic effects and to normalize or maintain methyl metabolism in the body. Furthermore, a role for policosanol has been shown to enhance or maintain brain function, nerve function, liver function, bone health, joint health, prostate health, fetal development, postnatal breast milk production, eye health and physical performance.

While various types of policosanol tablets or capsules are available, there remained an unmet need for a totally natural source of policosanol and food products containing that policosanol.

### SUMMARY OF THE INVENTION

The present invention provides a novel type of health food product that contains sugar cane juice. The sugar cane juice contains policosanol. The sugar cane juice is preferably extracted at a temperature below 150° C., more preferably less than 110° C., most preferably less than 105° C.

In another aspect of the invention, a method of decreasing total cholesterol and low density lipoprotein (LDL cholesterol) is provided. The method of the invention also increases HDL levels. The method comprises administering the equivalent of one teaspoon of sugar cane juice per day for several weeks. As used herein the terms “administering” and “administer” encompass self-administration or ingestion. The sugar cane juice may be provided in a variety of food products such as candies, puddings, beverages, and the like. Alternatively, straight sugar cane juice may be administered to the subjects.

In one aspect of the invention, a method of lowering LDL-cholesterol is provided. The method comprises administering to a mammal a daily dose of at least 1 g of sugar cane juice for at least one week. The method preferably comprises administering to a mammal a daily dose of at least 2 g, preferably at least 4 g of sugar cane juice for at least two weeks, more preferably four weeks. In a particularly preferred embodiment, about 5 g of sugar cane juice containing at least 50 mg of policosanol is ingested.

In a preferred embodiment, the sugar cane juice is provided in a composition comprising at least one additional agent selected from the group consisting of: amino acids, carbohydrates, vitamins, minerals, fats (preferably polyunsaturated fatty acids, more preferably omega-3 fatty acids), flavenols, sterols, psyllium, garlic, gugulipid, anti-oxidants, folic acid, beta-carotene, lycopene, red yeast rice extract, natural and synthetic flavors, colors, preservatives, stabilizers, emulsifiers, leavening agents and other processing aids. Combinations of additional additives may be included. In a preferred embodiment, the additional agent is a phytosterol. In a further preferred embodiment, the phytosterol is present in an amount from about 0.01 to about 20 g per day, preferably about 0.1 to 1.0 g/day.

In a preferred embodiment of the method, the sugar cane juice is provided for oral consumption in a food product selected from the group consisting of cake, cookie, confectionary, energy bar, pudding, yogurt, frozen dessert, pie, pastry, chewing gum, sauces, gravies, beverages including alcoholic beverages, and other processed foods. The invention applies to any application wherein sugar or another sweetener can be replaced by sugar cane juice containing policosanols. A preferred daily dose of sugar cane juice comprises at least 1 mg of policosanol. The daily dose is preferably administered for at least 4 weeks, more preferably for at least six weeks.

In a preferred embodiment, the sugar cane juice comprises from about 1 to about 100 mg of policosanol per 5 g of juice.

In an alternative embodiment, the sugar cane juice is provided in a format for intravenous administration.

In another aspect of the invention a food product is provided that comprises sugar cane juice having at least 1 mg policosanol per 5 g of sugar cane juice.

In one embodiment of the invention, the food product comprises an additional agent selected from the group consisting of: amino acids, carbohydrates, vitamins, minerals, fats (preferably polysaturated fatty acids, more preferably omega-3 fatty acids), flavenols, sterols, psyllium, garlic, gugulipid, anti-oxidants, folic acid, beta-carotene, lycopene, red yeast rice extract, natural and synthetic flavors, colors, preservatives, stabilizers, emulsifiers, leavening agents and other processing aids. Combinations of additional additives may be included. In a preferred embodiment, the additional agent is a phytosterol. In a further preferred embodiment, the phytosterol is present in an amount from about 0.01 to about 20 g per day, preferably about 0.1 to 5.0 g/day.

A further aspect of the invention provides for the use of sugar cane juice as an agent for the treatment or prevention of cardiovascular disease. The sugar cane juice typically comprises at least 5 mg policosanol per 5 g juice.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 illustrates the mean cholesterol for the base line and treatment groups;

FIG. 2 illustrates graphically the mean HDL cholesterol for the base line and treatment groups;

FIG. 3 displays graphically the mean LDL cholesterol for the base line and treatment scores.

### DETAILED DESCRIPTION

There is a growing demand for functional foods that couple health benefits with good taste. In particular, consumers are demanding healthier snack foods.

The present invention provides a food product comprising sugar cane juice as a source of policosanols. Policosanols are normally derived from sugar cane wax or beeswax. They have also previously been isolated from rice bran wax. All the previous sources were waxy substances. The policosanols are extracted through a series of steps and purified. Contrary to popular belief, the present inventors found the surprising result that sugar cane juice prepared without excess heating also contains therapeutically relevant levels of policosanol. The results of the analysis of one sample of sugar cane juice are discussed in Example 1 below. Sugar cane juice provides an all-natural source of unrefined policosanols that have proven health benefits. Using sugar cane juice as a source of policosanols provides the added advantage of providing an all-natural sweetener. This provides for a food product that combines good taste and heart-healthy supplements.

The invention also provides a method for the treatment or prevention of cardiovascular disease. As discussed further in Example 2 below and as illustrated in the attached Figures, total cholesterol and LDL cholesterol were significantly reduced in patients that ingested sugar cane juice containing policosanols on a daily basis. Mean HDL cholesterol was also increased.

Daily ingestion of about 0.1 to 20 g of sugar cane juice containing about 0.1 to 2000 mg policosanol is recommended. The sugar cane juice may be administered alone. Preferably, the sugar cane juice is incorporated into a snack food or beverage.

Policosanol is a term that refers to a group of natural products composed of various fatty alcohols. The food product according to this invention is especially a functional food, a nutritional supplement, a pharma food, a health food, a designer food, or any food product. A suitable amount of policosanol in the food product is from about 1 to 2000 mg of policosanols per serving. The functional food according to this invention can for example be in the form of candy, cookie, cake, chewing gum, yogurt, frozen dessert, or jelly, jams, pastry, pie filling, donuts, pudding, a beverage or the like. Other foods such as cereals, soups, gravies and prepared meals containing sugar cane juice as a source of policosanols are also encompassed. Any application where sugar cane juice can be used as a source of sugar is within the scope of the invention. This includes formulations for intravenous administration.

The food product of the invention comprising policosanol-containing sugar cane juice may also include other nutritional components. For example, dietary fiber and protein may be included in the food formulation. Other possible components include amino acids, carbohydrates, vitamins, minerals, fats, flavenols, sterols, psyllium, garlic, gugulipid, anti-oxidants, folic acid and the like. The composition may also include polyunsaturated fatty acids, particularly omega-3 fatty acids. Nutraceuticals such as beta-carotene, lycopene, and red yeast rice extract, etc. may be included. This includes any substance that is a food or a part of a food and provides medical or health benefits, including the prevention and treatment of disease. Such products may range from isolated nutrients, dietary supplements, herbal products, genetically engineered designer foods, stimulant functional foods or pharma foods.

Additional ingredients that may be incorporated include natural and synthetic flavors, colors, preservatives and stabilizers. To obtain a desired consistency, other agents such as emulsifiers, leavening agents and other processing aids may be included.

In one preferred embodiment, a food product comprising policosanol-containing sugar cane juice further comprises another cholesterol lowering agent. This includes an agent that reduces cholesterol adsorption, an agent that inhibits cholesterol biosynthesis and/or an agent that increases cholesterol metabolism.

One example of a preferred agent for use in the food product is phytosterol. Phytosterols are steroids derived from plants, yeast or fungi. They are preferably obtained from whole plants or parts thereof such as leaves, stalks, roots, shoots, rhizomes, fruits, kernels, husks, etc. Naturally occurring compositions or extracts may be used. The term “phytosterol” is used herein to refer to plant sterols and plant stanols that differ from cholesterol in that the side chain at position 17 contains a double bond and/or an additional methyl, ethyl or ethylidene group. The term also encompasses any analogues, derivatives or combinations thereof.

Phytosterols are similar to cholesterol and compete with cholesterol to get absorbed into the body. In the present invention, the phytosterols may be combined with lecithin to enhance their solubility and absorption. A mixture of more than one phytosterol may be included. In one embodiment, the phytosterol is one that reduces cholesterol absorption in the intestine. In another embodiment, the phytosterol inhibits cholesterol biosynthesis. An exemplary phytosterol composition for use in the present Invention could comprise a composition that inhibits the enzyme, 3-hydroxy-3-methylguaryl coenzyme A reductase (HMG+CoA reductase). In yet another embodiment, a phytosterol composition capable of increasing cholesterol metabolism is provided. The phytosterol increases the conversion of cholesterol into bile acids or inhibits the esterification of cholesterol. These may include, for example, chblesterol-7a hydroxylase activator or Acyl-CoA acyl transferase inhibitor. In a further preferred embodiment, a combination of phytosterols may be included with the policosanol-containing sugar cane juice.

A method for reducing blood serum cholesterol levels or preventing elevated blood cholesterol levels is provided. The method comprises administering or ingesting an effective amount of a composition comprising sugar cane juice. The method of the invention is effective in lowering total cholesterol, decreasing LDL-cholesterol and/or increasing HDL-cholesterol. The composition is preferably provided in the form of a food or beverage. The composition is preferably administered orally. A daily dose of the composition comprises from about 1 to about 2000 mg policosanol, preferably at least 10 mg policosanol, more preferably at least 20 mg, most preferably at least 40 mg policosanol. Alternatively, the composition may administered as a syrup. The composition can also be administered intravenously as a drip.

The method of the invention optionally includes the co-administration of one or more active agents in addition to the sugar cane juice. The additional active agents may be provided in the same composition as the sugar cane juice or in a separate composition. An additional active agent for use in the invention is preferably an active agent that reduces LDL cholesterol or increases HDL-cholesterol. A preferred additional active agent is a phytosterol.

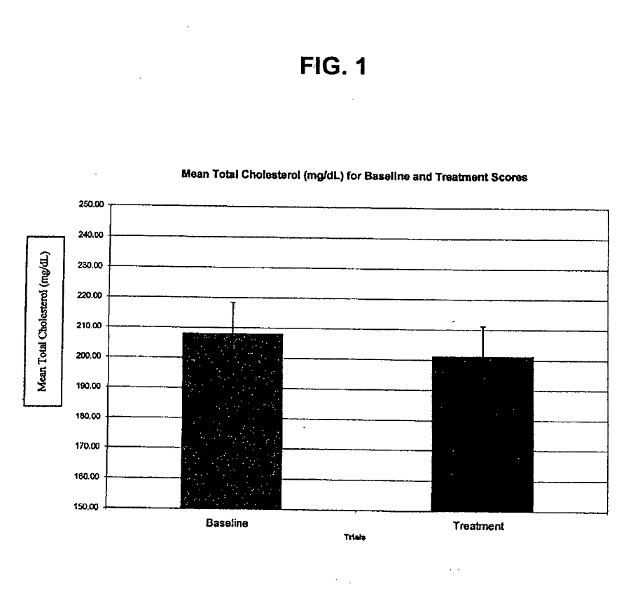

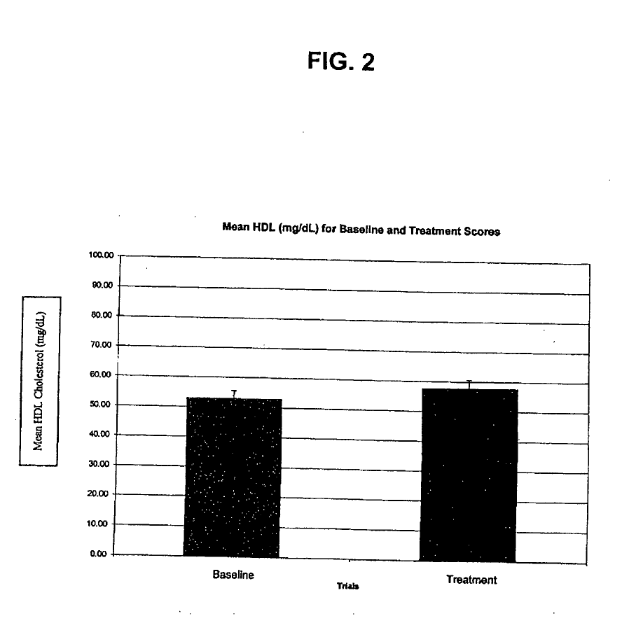

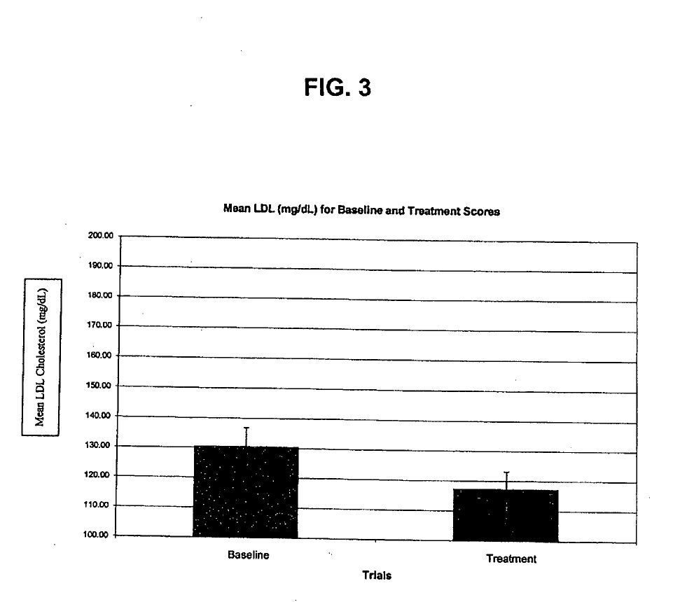

As shown in FIGS. 1 to 3 and discussed in greater detail in the Examples below, a daily dose of sugar cane juice significantly decreases overall cholesterol and specifically LDL cholesterol while causing an increase in HDL levels. FIG. 1 illustrates that mean total cholesterol levels were decreased after the treatment as compared to the baseline score. FIG. 2 illustrates that mean HDL levels increase as compared to the baseline after ingestion of sugar cane juice. FIG. 3 illustrates that the most significant effect the reduction of LDL scores when the levels post treatment are compared to the baseline levels.

The present invention demonstrates that a composition comprising sugar cane juice can have significant effects on cardiovascular health as measured by serum cholesterol levels. Without being limited by explanation, it is likely that these effects are due to policosanols in the sugar cane juice. Of course, it is considered that other heart healthy components could also be present in the sugar cane juice. The results presented herein demonstrate that sugar cane juice can contain clinically relevant amounts of policosanol. Daily administration of policosanol-containing sugar cane juice has been shown to be safe and tolerable in humans and lowers total cholesterol and LDL-cholesterol. Ingestion of sugar cane juice also elevates HDL levels. Policosanol may exert its effects by regulating the activity of HMG-CoA reductase and LDL-C processing. There is also some evidence that policosanol can inhibit in vitro platelet aggregation. Furthermore, policosanol has been associated with reduced in vivo oxidative stress in some studies and has been associated with an increase in exercise endurance in patients with intermittent claudication and coronary artery disease. These results indicate that compositions and methods including sugar cane juice as a source of policosanols will provide significant health and economic benefits.

The present invention also provides a method of preparing sugar cane juice that preserves health promoting active agents. In traditional methods, high temperatures are used in the extraction process. In the method of the present invention, the temperature of the processing system is maintained below 150° C., preferably below 120° C., more preferably below 105° C. Sugar can juice prepare according to the present invention contains policosanols that would normally be lost through a high temperature process.

The above disclosure generally describes the present invention. It is believed that one of ordinary skill in the art can, using the preceding description, make and use the compositions and practice the methods of the present invention. A more complete understanding can be obtained by reference to the following specific examples. These examples are described solely to illustrate preferred embodiments of the present invention and are not intended to limit the scope of the invention. Changes in form and substitution of equivalents are contemplated as circumstances may suggest or render expedient. Other generic configurations will be apparent to one skilled in the art. All journal articles and other documents such as patents or patent applications referred to herein are hereby incorporated by reference.

### EXAMPLES

Although specific terms have been used in these examples, such terms are intended in a descriptive sense and not for purposes of limitation. Methods of biochemistry and chemistry referred to but not explicitly described in the disclosure and these examples are reported in the scientific literature and are well known to those skilled in the art.

### Example 1

### Analysis of Sugar Cane Juice for Policosanols

The fatty acid content of a sample of sugar cane juice was determined by gas chromatography with flame ionization detection. The concentration of the policosanol marker compound (octacosanol) was calibrated using a sample from Sigma chemicals and the results for the other fatty alcohols were based on the octacosanol calibration. Standards for tetracosanol, hexacosanol and triacosanol were used as retention time markers. Results for dotriacontanol and tetratricontanol were based on predicted relative retention times. The results are shown in Table 1 below:

TABLE 1
ALPHA Sample #04-2653

| | | |
|---|---|---|
| Policosanol by GC-FID | Result | Units |

I. Octacosanol by GC-FID

| | | |
|---|---|---|
| Octacosanol | <0.5 | % wt/wt |
| Octacosanol (D) | <0.5 | % wt/wt |

II. Fatty Alcohols Identified by Comparison with Standards

| | | |
|---|---|---|
| Tetracosanol | <0.5 | % wt/wt |
| Tetracosanol (D) | <0.5 | % wt/wt |
| Hexacosanol | <0.5 | % wt/wt |
| Hexacosanol (D) | <0.5 | % wt/wt |
| Triacontanol | <0.5 | % wt/wt |
| Triacontanol (D) | | |

III. Tentatively Identified Fatty Alcohols

| | | |
|---|---|---|
| Dotriacontanol | <0.5 | % wt/wt |
| Dotriacontanol (D) | <0.5 | % wt/wt |
| Tetratriacontanol | 2.2 | % wt/wt |
| Tetratriacontanol (D) | 2.4 | % wt/wt |

Each result is the average of two injections per sample preparation.
(D) = Duplicate sample preparation for internal quality check.

### Example 2

### Effect of Daily Sugar Cane Juice on Cholesterol Levels

Twenty-three male and female participants of all ethnic backgrounds from the ages of 19-62 were randomly selected without screening. Subjects were instructed to fast on the mornings that blood was drawn, which occurred between the hours of 7:00-9:30 a.m. Conducting the investigation as a single-blind study, an initial blood sample was taken and tested for complete lipid panels. Each participant was given two bottles of sugar can juice product containing 100 mg of policosanols per 5 g serving. Each subject was instructed to ingest 1 teaspoon (5 g) each day for an eight-week duration. A final blood sample was then drawn for lipid panels. A within groups dependent T-test on difference scores was run on the data to ascertain the effects on total cholesterol, HDL cholesterol, and LDL cholesterol.

A total of 23 participants chose to participate in this study; however, 5 subjects were disqualified based on the following post-test questionnaire responses: noncompliance to take the product every day for the full eight week duration (3 subjects)—and in absentia during the final blood drawing (2 subjects). No side effects were reported for any participants.

For the remaining qualifying subjects, Table 2 below displays the mean values for total cholesterol, HDL cholesterol, and LDL cholesterol for the baseline and treatment groups. The percent increase or decrease was also noted for both trials. The mean values of total cholesterol for the baseline and treatment scores were 208.00 and 201.11, respectively, indicating a 3.31% decrease from baseline to treatment scores. The mean values of HDL cholesterol for the baseline and treatment scores were 52.89 and 57.67, respectively, signifying an increase of 8.29% from baseline to treatment scores. The mean values of LDL cholesterol for the baseline and treatment scores were 130.39 and 117.28, respectively, showing a 10.40% decrease from baseline to treatment scores.

TABLE 2

| | Mean Baseline | Mean Treatment | |
|---|---|---|---|
| Measurement | Score | Score | Percent Change |
| Total Cholesterol | 208.00 | 201.11 | −3.31% |
| (mg/dL) | | | |
| HDL Cholesterol | 52.89 | 57.67 | +8.29% |
| (mg/dL) | | | |
| LDL Cholesterol | 130.89 | 117.28 | −10.40% |
| (mg/dL) | | | |

FIG. 1 shows the mean cholesterol for the baseline and treatment trials. Standard error bars show some overlap between trials; however, the dependent samples t-Test showed a significant decrease in total cholesterol in the treatment reading over the baseline reading: t (17)=2.09, p<0.05. Mean total cholesterol decreased 3.31% from the baseline reading to the treatment reading.

FIG. 2 shows the mean HDL cholesterol for the baseline and treatment trials. Standard error bars show little overlap in the data, and the dependent samples t-Test reported the treatment scores having significantly increased HDL cholesterol than baseline scores: t(17)=2.07, p<0.05. Mean HDL cholesterol increased 8.29% from the baseline reading to the treatment reading.

FIG. 3 displays the mean LDL cholesterol for the baseline and treatment scores. Standard error bars show little overlap in the data, and the dependent samples t-Test reported a significant decrease in LDL cholesterol for the treatment scores over the baseline scores: t(17)=3.01, p<0.05. Mean LDL cholesterol showed a 10.40% decrease from the baseline reading to the treatment reading.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A method of lowering LDL-cholesterol or increasing HDL-cholesterol comprising administering to a mammal a daily dose of at least 1 g of sugar cane juice for at least one week.

2. A method of lowering LDL-cholesterol or increasing HDL-cholesterol comprising administering to a mammal a daily dose of at least 1 g of sugar cane juice for at least two weeks.

3. A method of lowering LDL-cholesterol or increasing HDL-cholesterol comprising administering to a mammal a daily dose of at least 1 g of sugar cane juice for at least four weeks.

4. A method of lowering LDL-cholesterol or increasing HDL-cholesterol comprising administering to a mammal a daily dose of at least 1 g of sugar cane juice for at least six weeks.

5. A method according to claim 1, wherein said sugar cane juice comprises policosanol.

6. A method according to claim 1, wherein said sugar cane juice is provided in a composition comprising at least one additional agent selected from the group consisting of: amino acids, carbohydrates, vitamins, minerals, polysaturated fatty acids, flavenols, sterols, psyllium, garlic, gugulipid, anti-oxidants, folic acid, beta-carotene, lycopene, red yeast rice extract, natural and synthetic flavors, colors, preservatives, stabilizers, emulsifiers, leavening agents and combinations thereof.

7. A method according to claim 6 wherein said additional agent is a phytosterol.

8. A method according to claim 7 wherein said phytosterol is present in an amount from about 0.01 to about 20 g per day.

9. A method according to claim 7 wherein said phytosterol is present in an amount of at least 0.1 g.

10. A method according to claim 7 wherein said phytosterol is present in an amount of at least 1 g.

11. A method according to any one of the previous claims wherein said sugar cane juice is provided for oral consumption in a food product selected from the group consisting of: cake, cookie, candy, energy bar, pudding, yogurt, frozen dessert, pie, pastry, jam, jelly, pie filling, donuts, sauces, gravies, beverages, including alcohol beverages, chewing gum, and other processed foods.

12. A method according to claim 1 wherein the daily dose comprises at least 3 g of sugar cane juice.

13. A method according to claim 1 wherein the daily dose comprises at least 5 g of sugar cane juice.

14. A method according to claim 6 wherein the daily dose of sugar cane juice comprises at least 0.1 mg of policosanol.

15. A method according to claim 14 wherein the daily dose of sugar cane juice comprises at least 0.1 mg of policosanol.

16. A method according to claim 14 wherein the daily dose of sugar cane juice comprises at least 1 mg of policosanol.

17. A method according to claim 14 wherein the daily dose of sugar cane juice comprises at least 4 mg of policosanol.

18. A method according to claim 1 wherein the sugar cane juice is provided in a format for intravenous administration.

19. A method according to claim 1 wherein the sugar cane juice is extracted using a process wherein the temperature is maintained at less than 120° C.

20. A method according to claim 1 wherein the sugar cane juice is extracted using a process wherein the temperature is maintained at less than 105° C.

21. A composition for lowering LDL cholesterol, said composition comprising sugar cane juice having at least 0.1 mg policosanol.

22. A composition according to claim 21 wherein the sugar can juice comprises at least 0.5 mg policosanol.

23. A composition according to claim 21 wherein the sugar can juice comprises at least 1 mg policosanol.

24. A composition according to claim 21 wherein the sugar can juice comprises at least 2 mg policosanol.

25. A composition according to claim 21 wherein the sugar can juice comprises at least 4 mg policosanol.

26. A composition according to claim 18, wherein said composition includes an additional agent selected from the group consisting of dietary fiber, protein, amino acids, carbohydrates, vitamins, minerals, fats, flavenols, sterols, psyllium, garlic, gugulipid, anti-oxidants, folic acid, polyunsaturated fatty acids, omega-3 fatty acids, beta-carotene, lycopene, red yeast rice extract, synthetic flavors, colors, preservatives, stabilizers, emulsifiers, and leavening agents.

27. A composition according to claim 21, wherein said additional agent is a phytosterol.

28. A composition according to claim 26 wherein the phytosterol is provided in an amount of at least 0.1 g.

29. A composition according to claim 26 wherein the phytosterol is provided in an amount of at least 1 g.

30. A composition according to claim 26 wherein the phytosterol is provided in an amount of at least 5 g.

31. An LDL-lowering food product comprising sugar cane juice as an active agent, whereby daily consumption of the food product results in a decrease in LDL levels relative to the baseline level.

32. A food product comprising sugar cane juice as an agent to increase HDL levels.

33. Use of sugar cane juice as an agent for the treatment or prevention of cardiovascular disease.

34. Use according to claim 31, wherein said juice comprises at least 1 mg policosanol.

35. A method of preparing sugar cane juice containing policosanols comprising extracting the juice from sugar cane at a temperature of less than 120° C.

36. A method of preparing sugar cane juice containing policosanols comprising extracting the juice from sugar cane at a temperature of less than or equal to 105° C.
